(19) United States
(12) Patent Application Publication
(10) Pub. No.: US 2022/0142601 A1
(43) Pub. Date: May 12, 2022

(54) FLEXIBLE PATCH FOR RESPIRATORY SOUND MONITORING

(71) Applicant: Hunan VentMed Medical Technology Co., Ltd., Shaoyang (CN)

(72) Inventors: Jinping SU, Shaoyang (CN); Zaixin YUAN, Shaoyang (CN); Qichang ZHENG, Shaoyang (CN)

(21) Appl. No.: 17/394,448

(22) Filed: Aug. 5, 2021

(30) Foreign Application Priority Data
Nov. 6, 2020 (CN) 2020112262886

(51) Int. Cl. A61B 7/00; A61B 5/00

(52) U.S. Cl. CPC A61B 7/003; A61B 2562/16; A61B 2562/0214; A61B 5/6833

(57) ABSTRACT

A flexible patch for respiratory sound monitoring, comprising an acceptance box, an acceptance motor, an operation screen, a storage box, the flexible patch, a framework, a hand lever, a top cover, a detection range telescopic rod. The present disclosure provides the acceptance box, the storage box, the acceptance motor and an acceptance shaft to realize the purpose of convenient storage and access of flexible patches to prevent damage. And the flexible patch improves the measurement accuracy, the detection range telescopic rod that is connected with the upper part of the respiratory sound receiving sensor control the sensor slides along the slide rail to facilitate the adjustment of a sensor's monitoring range, and the respiratory sound horn amplifier set the lower part of the respiratory sound receiving sensor improves the detection effect of the sensor.

### CROSS-REFERENCE TO RELATED APPLICATIONS

The application claims priority to Chinese patent application No. 202011226288.6, filed on Nov. 6, 2020, the entire contents of which are incorporated herein by reference.

### TECHNICAL FIELD

The present disclosure belongs to the field of medical equipment, and particularly to a flexible patch for respiratory sound monitoring.

### BACKGROUND

Respiratory sounds are sounds that flow through the respiratory tract and alveoli when a normal person breathes, generating turbulence, causing vibrations and making the sounds, which are transmitted to the body surface through the lung tissue and chest wall. A doctor can check a patient's physical condition by monitoring the intensity, pitch, nature, length of the phase of the respiratory sound and the patient's auscultation site.

The traditional respiratory sound flexible patch is too soft and inconvenient to take when it is used, so that the user cannot accurately stick to the particular body part when sticking to the body, and adhesive patches are also easy to stick together, and the data transmission line connected by the flexible patch is long and easy to be enwound together, therefore, the traditional respiratory sound flexible patch is inconvenient to use.

The patent with publication number CN210408417U provides a fetal heart rate monitoring patch, and the fetal heart rate monitoring patch includes multiple electrode patches, a connector and a measuring device, and the electrode patches are connected with the connector through a wire. When the fetal heart rate monitoring patch is storing information or using, the wire is so long that it will be enwound, and the electrode patch is inconvenient for users to take and use.

Therefore, it is very necessary to invent a flexible patch that can be conveniently stored by the user and is convenient for the user to accurately paste the detected respiratory sound monitoring.

### SUMMARY

To solve the problem alleged in the background technology, the present disclosure provides a flexible patch for respiratory sound monitoring, and the flexible patch can be conveniently stored by the user and is convenient for the user to accurately paste.

The flexible patch for respiratory sound monitoring, comprising:

the flexible patch comprises an acceptance box, an acceptance motor, an operation screen, a storage box, the flexible patch, a framework, a hand lever, a top cover, a detection range telescopic rod; the acceptance motor is fixedly installed on the side of the acceptance box; the rear of the acceptance box is fixedly connected with the operation screen; the storage box can be stored into the acceptance box; the flexible patch is placed inside the acceptance box; the upper part of the flexible patch is fixedly connected with the framework; the upper part of the framework is fixedly provided with the top cover; the upper part of the top cover is fixedly connected with the hand lever; and the upper part of the flexible patch is connected with the detection range telescopic rod.

Further, the bottom of the acceptance box is fixedly provided with a base, the side of the acceptance box is fixedly connected with a handle, and the acceptance box contains a data transmission line inside.

Further, the acceptance box comprises an acceptance groove, and the front side of a bottom of the acceptance box is provided with an acceptance groove.

Further, the acceptance motor includes an acceptance shaft, and the acceptance shaft is connected with the acceptance motor in rotation.

Further, the operation screen includes a retaining bracket, and the operation screen is fixedly connected with the acceptance box through the retaining bracket on both sides.

Further, the upper part of the flexible patch is fixedly equipped with a data connection axis, and the upper part of the hand lever is provided with a detection range telescopic controller with the flexible patch.

Further, the flexible patch includes a respiratory sound receiving sensor, a respiratory sound horn amplifier and a sensor slide rail; three respiratory sound receiving sensors are fixed at a bottom of the flexible patch; several respiratory sound horn amplifiers are arranged on the upper part of the respiratory sound receiving sensor; and the sensor slide rails are fixed on both sides of the flexible patch; and the respiratory sound receiving sensor is a CNZ-15 electret capacitive sensor.

Further, the framework includes a coupling shaft; and the flexible patch is fixedly connected to a bottom of the framework through the coupling shaft.

Further, the top cover includes a magnetic link block, and the magnetic link block is fixedly installed at a bottom of the top cover.

Further, the detection range telescopic rod includes a rotating shaft and a lower shaft; the upper part of the detection range telescopic rod is rotatably connected with the rotating shaft; and the lower part of the detection range telescopic rod is rotatably connected with the lower shaft.

Compared with the prior art, the beneficial effects of the present disclosure are the following:

1. Prevent damage to the flexible patch: the disclosure is provided with an acceptance box and an acceptance motor, and an acceptance shaft is connected with the side of the acceptance motor, which can rotate a longer data transmission line. Put the data transmission line out when people need to use it; and the storage box and the acceptance groove are convenient to store the flexible patch to prevent damage to the flexible patch.

2. Controllable detection range: the upper part of the internal flexible patch is provided with a detection range telescopic rod, and the detection range telescopic rod is connected with the respiratory sound receiving sensor, and the detection range telescopic rod can adjust the respiratory sound receiving sensor to slide left and right along the sensor slide rail to adjust the detection range of the respiratory sound receiving sensor.

3. Physical amplification of the respiratory sound to improve detection accuracy: several respiratory sound horn amplifiers are arranged on the upper part of the respiratory sound receiving sensor, and the received respiratory sound can be amplified through the contact between the respiratory sound horn amplifier and the patient's body. In doing so, it is convenient to receive the respiratory sound from the respiratory sound receiving sensor, so that improve the detection accuracy.

4. The use of the flexible patch: there is a hand lever on the upper part of the flexible patch, which is convenient for the user to attach the flexible patch to the accurate position of the body, improving the detection accuracy; and the framework will prevent the flexible patch from sticking together which is convenient for users to use.

5. Convenient to carry: the flexible patch can be completely stored in the acceptance box, minimizing the size of the device and making it convenient for medical staff to transfer the device.

6. Convenient to operate: the upper part of the acceptance box is equipped with an operation screen, and the side is equipped with a handle, which is convenient for the user to hold the device and achieve the effect of convenient use of the device.

7. Prevent the data transmission line from enwinding: the acceptance motor and acceptance shaft can wind up the data transmission line when not in use to prevent the data transmission line from enwinding causing inconvenience for subsequent use.

### BRIEF DESCRIPTION OF DRAWINGS

FIG. 1 illustrates a flexible patch schematic diagram of the present disclosure;

FIG. 2 shows schematic diagram of the present disclosure when in use;

FIG. 3 shows a main view of the present disclosure when in use;

FIG. 4 illustrates schematic diagram of the acceptance structure of the present disclosure.

### DETAILED DESCRIPTION OF THE EMBODIMENTS

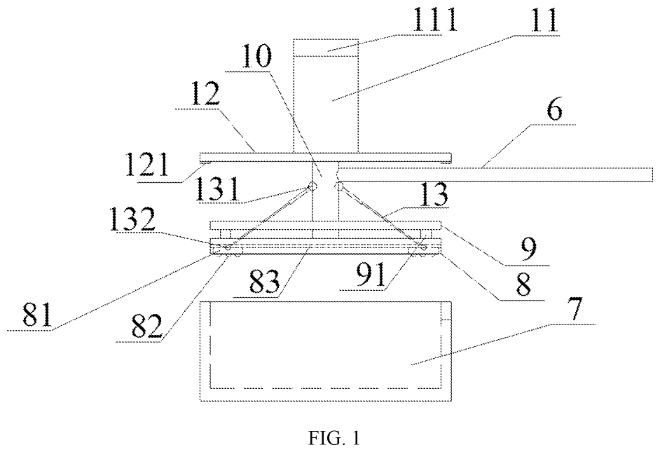

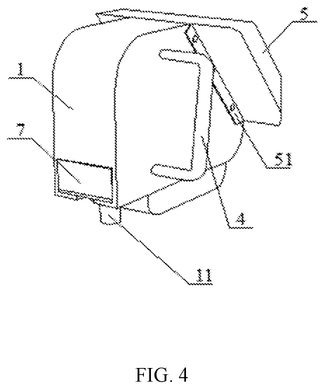

Refer to FIG. 1-FIG. 4, the flexible patch for respiratory sound monitoring, and the flexible patch comprises an acceptance box 1, an acceptance motor 2, an operation screen 5, a storage box 7, the flexible patch 8, a framework 9, a hand lever 11, a top cover 12; the acceptance motor 2 is fixedly installed on the side of the acceptance box 1; the rear of the acceptance box 1 is fixedly connected with the operation screen 5; the storage box 7 can be stored into the acceptance box 1; the flexible patch 8 is placed inside the acceptance box 1; the upper part of the flexible patch 8 is fixedly connected with the framework 9; the upper part of the framework 9 is fixedly provided with the top cover 12; the upper part of the top cover 12 is fixedly connected with the hand lever 11; and the upper part of the flexible patch 8 is connected with the detection range telescopic rod 13.

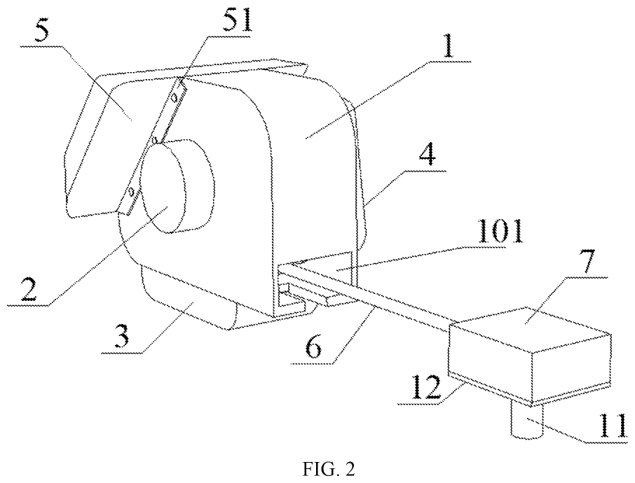

Refer to FIG. 2, a bottom of the acceptance box 1 is fixedly provided with a base 3, the side of the acceptance box 1 is fixedly connected with a handle 4, and the acceptance box 1 contains a data transmission line 6 inside.

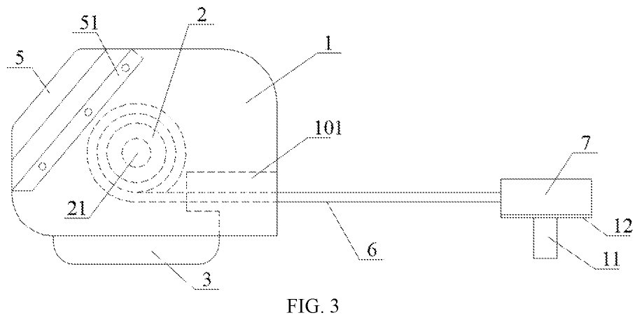

Refer to FIG. 2-FIG. 3: the acceptance box 1 comprises an acceptance groove 101, and the front side of a bottom of the acceptance box 1 is provided with an acceptance groove 101.

Refer to FIG. 3, the acceptance motor 2 includes an acceptance shaft 21, and the acceptance shaft 21 is connected with the acceptance motor 2 in rotation.

Refer to FIG. 2-FIG. 4, the operation screen 5 includes a retaining bracket 51, and the operation screen 5 is fixedly connected with the acceptance box 1 through the retaining bracket 51 on both sides.

Refer to FIG. 1-FIG. 4, the upper part of the flexible patch 8 is fixedly equipped with a data connection axis 10, and the upper part of the hand lever 11 is provided with a detection range telescopic controller 111 with the flexible patch 8.

The above embodiments, further: the flexible patch 8 includes a respiratory sound receiving sensor 81, a respiratory sound horn amplifier 82 and a sensor slide rail 83; three respiratory sound receiving sensors 81 are fixed at a bottom of the flexible patch 8; several respiratory sound horn amplifiers 82 are arranged on the upper part of the respiratory sound receiving sensor 81; and the sensor slide rails 83 are fixed on both sides of the flexible patch 8; and the respiratory sound receiving sensor 81 is a CNZ-15 electret capacitive sensor.

Further, the framework 9 includes a coupling shaft 91; and the flexible patch 8 is fixedly connected to a bottom of the framework 9 through the coupling shaft 91.

Further, the top cover 12 includes a magnetic link block 121, and the magnetic link block 121 is fixedly installed at a bottom of the top cover 12.

Further, the detection range telescopic rod 13 includes a rotating shaft 131 and a lower shaft 132; the upper part of the detection range telescopic rod 13 is rotatably connected with the rotating shaft 131; and the lower part of the detection range telescopic rod 13 is rotatably connected with the lower shaft 132.

When the present disclosure is in use, starting and counterclockwise rotating the acceptance motor 2 with the data transmission line 6 wound on the upper part of the acceptance shaft 21 and releases acceptance motor 2 with the data transmission line 6 from the acceptance box 1.

Firstly, the user takes the hand lever 11 and takes the flexible patch 8 out of the storage box 7, then grabs the flexible patch 8 by the hand lever 11 and stick the flexible patch 8 to the body to detect respiratory sounds. At this time, the respiratory sound receiving sensor 81 starts to work to receive respiratory sound information and transmit the collected information to the operation screen 5 through the data connection axis 10 and the data transmission line 6 for analysis and display.

Secondly, through the detection range telescopic controller 111 set on the upper part of the handle 11, controlling the detection range telescopic rod 13 to contract, and the rotating shaft 131 and the lower shaft 132 rotate accordingly. The respiratory sound receiving sensor 81 connected with the detection range telescopic controller 111 slides inward along the sensor slide rail 83. At the time, the detection position of the respiratory sound receiving sensor 81 can be adjusted to expand the detection range and improve the detection effect.

After the detection is completed, the flexible patch 8 is restored to its original position through the detection range telescopic controller 111. Then put the flexible patch 8 into the storage box 7 and connect the flexible patch 8 with the upper side of the storage box 7 through the magnetic link block 121 at the bottom of the top cover 12. The data transmission line 6 is placed in the notch on the side of the storage box 7, and then start the acceptance motor 2, and the acceptance shaft 21 rotates clockwise to wind the data transmission line 6 onto the acceptance shaft 21, and the data transmission line 6 is stored in the acceptance box 1, and the storage box 7 is inserted in the acceptance groove 101.

In the description of the present disclosure, it should be understood that the terms “upper”, “lower”, “front”, “rear”, “left”, “right”, “top”, “bottom”, “inner”, “etc. mean that positional relationship is based on the azimuth or positional relationship shown in the drawings. It is only for the convenience of describing the present disclosure and simplifying the description, rather than indicating or implying that the device or element referred to must have a specific orientation and be constructed and operated in a specific orientation, and therefore cannot be understood as a limitation of the present disclosure.

Finally, it should be noted that the above embodiments are only the preferred embodiments of the present disclosure and are not intended to limit the present disclosure, although the present disclosure has been described in detail with reference to the foregoing embodiments. For those skilled in the art, they can still modify the technical solutions described in the foregoing embodiments, or equivalently replace some of the technical features, and any modification, equivalent replacement, improvement, etc. made within the spirit and principle of the present disclosure shall be included in the protection scope of the present disclosure.

1. A flexible patch for respiratory sound monitoring, wherein:
the flexible patch comprises an acceptance box, an acceptance motor, an operation screen, a storage box, the flexible patch, a framework, a hand lever, a top cover, and a detection range telescopic rod; the acceptance motor is fixedly installed on a side of the acceptance box; a rear of the acceptance box is fixedly connected with the operation screen; the storage box can be stored into the acceptance box; the flexible patch is placed inside the acceptance box; an upper part of the flexible patch is fixedly connected with the framework; an upper part of the framework is fixedly provided with the top cover; an upper part of the top cover is fixedly connected with the hand lever; the upper part of the flexible patch is connected with the detection range telescopic rod, the acceptance motor is operationally connected to a data transmission line, and the data transmission line is operationally connected to the flexible patch.

2. The flexible patch for respiratory sound monitoring according to claim 1, wherein:
a bottom of the acceptance box is fixedly provided with a base, the side of the acceptance box is fixedly connected with a handle, and the acceptance box comprises the data transmission line inside.

3. The flexible patch for respiratory sound monitoring according to claim 1, wherein:
the acceptance box comprises an acceptance groove, and a front side of a bottom of the acceptance box is provided with the acceptance groove.

4. The flexible patch for respiratory sound monitoring according to claim 1, wherein:
the acceptance motor includes an acceptance shaft, and the acceptance shaft is connected with the acceptance motor in rotation.

5. The flexible patch for respiratory sound monitoring according to claim 1, wherein:
the operation screen includes a retaining bracket, and the operation screen is fixedly connected with the acceptance box through the retaining bracket on both sides.

6. The flexible patch for respiratory sound monitoring according to claim 1, wherein:
the upper part of the flexible patch is fixedly equipped with a data connection axis, and an upper part of the hand lever is provided with a detection range telescopic controller with the flexible patch.

7. The flexible patch for respiratory sound monitoring according to claim 1, wherein:
the flexible patch includes a respiratory sound receiving sensor, a respiratory sound horn amplifier and a sensor slide rail; three respiratory sound receiving sensors are fixed at a bottom of the flexible patch; several respiratory sound horn amplifiers are arranged on an upper part of the respiratory sound receiving sensor; and the sensor slide rails are fixed on both sides of the flexible patch; and the respiratory sound receiving sensor is a CNZ-15 electret capacitive sensor.

8. The flexible patch for respiratory sound monitoring according to claim 1, wherein:
the framework includes a coupling shaft; and the flexible patch is fixedly connected with a bottom of the framework through the coupling shaft.

9. The flexible patch for respiratory sound monitoring according to claim 1, wherein:
the top cover includes a magnetic link block, and the magnetic link block is fixedly installed at a bottom of the top cover.

10. The flexible patch for respiratory sound monitoring according to claim 1, wherein:
the detection range telescopic rod includes a rotating shaft and a lower shaft; an upper part of the detection range telescopic rod is rotatably connected with the rotating shaft; and a lower part of the detection range telescopic rod is rotatably connected with the lower shaft.

11. The flexible patch for respiratory sound monitoring according to claim 1, wherein:
the data transmission line is operationally connected to the flexible patch via a data connection axis.
